(19) United States
(12) Patent Application Publication
(10) Pub. No.: US 2004/0109879 A1
(43) Pub. Date: Jun. 10, 2004

(54) Method of Treating Herpes Virus Infections

(75) Inventors: Thomas B. Freeman, Tampa, FL (US); G. Michael Nauert, Tarpon Springs, FL (US)

Correspondence Address: SMITH & HOPEN PA, 15950 BAY VISTA DRIVE, SUITE 220, CLEARWATER, FL, 33760

(73) Assignee: UNIVERSITY OF SOUTH FLORIDA, Tampa, FL (US)

(21) Appl. No.: 10/604,591

(22) Filed: Aug. 1, 2003

Related U.S. Application Data
(60) Provisional application No. 60/319,442, filed on Aug. 1, 2002.

(51) Int. Cl. A61K 39/205

(52) U.S. Cl. 424/224.1

(57) ABSTRACT

The present invention is a novel alternative use of the rabies vaccine for the purposes of suppressing herpes outbreaks.

### CROSS REFERENCE TO RELATED APPLICATIONS

This application claims priority to provisional application 60/319,442, “Method of Treating Herpes Virus Infections”, filed Aug. 1, 2002.

### BACKGROUND OF INVENTION

Herpes virus infections are a recurring untreatable infectious problem with widespread epidemiological significance. Medical therapies are at best palliative. Currently, vaccine trials against Type 1 herpes (oral form) are only partially successful. There are no vaccines that are curative for either Type 1 or Type 2 (genital) herpes. The development of any therapy that provides long-lasting remission would therefore be of important clinical relevance.

There is evidence that the rabies virus glycoprotein cross-reacts with other viral glycoproteins. The rabies vaccine induces cross-reacting antibodies between the rabies virus and the human immunodeficiency virus-1 GP120. Both the HIV virus and the rabies virus share binding sites that are quite similar involving the nicotinic receptors on the viral surface. No such cross-reacting antibodies however have been described between vaccines against the rabies virus and the herpes virus. There are no previous clinical case reports on a similar cross-reaction.

It is, therefore, to the effective resolution of the aforementioned problems and shortcomings of the prior art that the present invention is directed. However, prior art references on both herpes and rabies do not anticipate or suggest the application of a rabies vaccine for the treatment of the herpes virus.

However, in view of the prior art in at the time the present invention was made, it was not obvious to those of ordinary skill in the pertinent art how the identified needs could be fulfilled.

### SUMMARY OF INVENTION

In a preferred embodiment, the present invention provides administration of a rabies vaccine to a patient for the treatment of a herpesviridae virus infection. Examples of viral infection included in the family of herpesviridae are infections from a herpes simplex type I or type II virus, a varicellovirus (zoster), cytomegalovirus, muromegalovirus, roseolovirus, lymphocrytpovirus, rhadinovirus, Epstein Barr virus, human herpes type 6 or type 7, and other unclassified viruses within the herpesviridae family of viruses.

In accordance with one embodiment of the invention, the vaccine is administered for the treatment of herpes simplex type 1.

In accordance with an additional embodiment of the invention, the vaccine is administered for the treatment of simplex type 2.

In a further embodiment, the rabies vaccine administered is a vaccine obtained from a human diploid cell, a purified chick embryo cell culture, an adsorbed vaccine, a pasteurized immunoglobulin vaccine, or an inactivated virus wherein the inactivation is done by heat, acid or beta propriolactone such as IMOVAX. IMOVAX is a human diploid cell vaccine manufactured by Aventis Pasteur. However, it would be clear to one of ordinary skill in the art that other rabies vaccines can be used and is within the scope of this invention.

In an additional embodiment, the rabies vaccine is administered intradermally yet another embodiment, the rabies vaccine is administered intramuscularly.

In an additional embodiment, the vaccine is administered on an as needed basis or as warranted. For example, conditions which would warrant a subsequent injection of the rabies vaccine will be when there is a re-occurrence of the herpes outbreak. This may occur when the initial efficacy of the initial injection of the vaccine has subsided. However, remission of herpes outbreak post treatment with the rabies vaccine will vary from patient to patient. Thus, it would be clear to one skilled in the art how often repeated injections of the vaccine may be applied. Administration of the vaccine can occur every 2 years, 3 years, 4 years, 5 years, or as necessary based on the diagnosis of herpes outbreak post vaccination.

In accordance with a preferred embodiment, a method of treating a patient suffering from herpes simplex type 1 provides for administering IMOVAX or an equivalent invactivated rabies vaccine to a patient on an as needed basis as described supra.

In an additional embodiment, a method of treating a patient suffering from herpes simplex type 2 provides for systemically administering IMOVAX to the patient or an equivalent invactivated rabies vaccine to a patient on an as needed basis as described supra.

In accordance with the present invention, a medicinal composition for the treatment of herpesviridae virus infections is provided, the composition comprising a rabies vaccine.

### DETAILED DESCRIPTION

The present invention relates to viral infections of the herpesviridae family, including in particular herpes simplex type 1 and herpes simplex type 2. More particularly, the present invention relates to a method and composition for the alleviation and control of such infections.

A rabies vaccine has the unintended capacity to induce cross-reacting antibodies that also suppress the herpes virus.

The following is a summary of the findings of a number of case histories demonstrating the effects of the method described by the present invention.

Patient #1 is a 65-year old male with long-term chronic oral herpes outbreaks, occurring approximately every two months. Due to a local rabies outbreak near his home, he received a rabies vaccine approximately eight years ago. Following that vaccine, all outbreaks ceased immediately for approximately two years. He had a subsequent boost for his rabies vaccine about 2½ years after his initial vaccination and once again the outbreaks of oral herpes ceased for about two years. A third boost was given approximately 2½ years later with similar result.

Patient #2 is Patient #1's wife. She is currently 37 years old. She had genital herpes, presumably obtained before marriage, as the lesion at the time of marriage in the early 1990's was a secondary rather than a primary lesion. Because of the fact that this lesion was present before marriage, it is not necessarily the same viral strain that was seen in Patient #1 and may indeed represent a herpes Type 2 lesion. She received a single rabies vaccine, also approximately seven years ago and has not had a single outbreak since that time.

Patient #3 is a woman who is now 27 years old. She is the babysitter for Patient #1. Five years ago, she had a history of numerous episodes of oral herpes recurring on a monthly basis. She received her first rabies vaccine five years ago, which provided complete remission of her oral herpes outbreaks. This lasted for approximately two years. The patient has subsequently had two rabies booster shots with remission provided for approximately two years after the first booster and remission is currently complete since the second booster approximately one year ago.

All three patients described above received vaccines from the same manufacturer, Aventis Pasteur, Inc. (Imovax rabies vaccine, administered intradermally). However, it is within the scope of the present invention to administer other rabies vaccines containing the cross-reacting material necessary to target the surface protein of the virus, either intradermally or intramuscularly.

Remission of the herpes virus was observed in all three patients. Previous to treatment, all three patients had frequent outbreaks on a monthly to bimonthly basis, with a dramatic change in the natural history of their disease. The rabies vaccines have previously been known to only provide benefit for approximately two years. However, the pharmacology of the vaccine will clearly vary from patient to patient. For example, Patient #2 from above had remission of herpes virus beyond two years post injection.

The invention would therefore be used on as needed basis, administered according to FDA guidelines as is current with medical practice.

The invention therefore is the alternative use of the rabies vaccine for the purpose of suppressing either Herpes Simplex Type 1 or Type 2 outbreaks.

It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

1. A method for treating herpesviridae virus infections, comprising administering a rabies vaccine to a patient.

2. The method of claim 1, wherein the herpesviridae virus is herpes simplex type 1.

3. The method of claim 1, wherein the herpesviridae virus is herpes simplex type 2.

4. The method of claim 1 wherein the rabies vaccine is selected from the group consisting of human diploid cell vaccine, purified chick embryo cell culture vaccine, rabies vaccine adsorbed vaccine, an inactivated rabies virus vaccine, a beta priopriolactine inactivated rabies virus vaccine, IMOVAX, and equivalent rabies vaccines thereof.

5. The method of claim 1, wherein the vaccine is administered intradermally.

6. The method of claim 1, wherein the vaccine is administered intramuscularly.

7. The method of claim 1, wherein the vaccine is administered on an as needed basis.

8. A method of treating a patient suffering from herpes simplex type 1, comprising administering a beta propriolactone inactivated rabies virus vaccine to the patient on an as needed basis.

9. A method of treating a patient suffering from herpes simplex type 2, comprising administering a beta propriolactone inactivated rabies virus vaccine to the patient on an as needed basis.
